(19) United States
(12) Patent Application Publication
(10) Pub. No.: US 2016/0256380 A1
(43) Pub. Date: Sep. 8, 2016

(54) USE OF NATURALLY-OCCURRING BLOOMS CONTAINING TOXIC CYANOBACTERIA

(71) Applicant: Stephanie Ann Smith, Columbus, OH (US)

(72) Inventor: Stephanie Ann Smith, Columbus, OH (US)

(73) Assignee: Beagle Bioproducts, Inc., Columbus, OH (US)

(21) Appl. No.: 15/153,251

(22) Filed: May 12, 2016

Related U.S. Application Data
(63) Continuation-in-part of application No. 14/511,510, filed on Oct. 10, 2014, now abandoned.
(60) Provisional application No. 61/889,349, filed on Oct. 10, 2013.

(51) Int. Cl. A61K 8/99; A61Q 19/10; A61Q 1/00; B01D 21/01; A61Q 5/12; A61Q 17/04; C12Q 1/04; A61Q 19/00; A61Q 19/08

(52) U.S. Cl. CPC A61K 8/99; A61Q 19/00; A61Q 19/10; A61Q 19/007; A61Q 1/00; A61Q 19/08; A61Q 5/12; A61Q 17/04; C12Q 1/04; B01D 21/01; A61K 2800/805

(57) ABSTRACT

Described herein are systems, methods and embodiments associated with the use of biomass for generating a preparation that can be used in manufacture of a personal care product. The described embodiments include collection of evidence of toxic cyanobacteria presence within biomass. Upon identification of biomass containing toxic cyanobacteria, embodiments describe processing of the biomass which will ultimately lead to the preparation. Thereafter, regardless of the initial presence of toxins, once the biomass is processed, the preparation can be safely used within a personal care product.

### CROSS REFERENCE TO RELATED APPLICATION

This patent application is a continuation-in-part of co-pending U.S. patent application Ser. No. 14/511,510 entitled “OBTAINING PRODUCTS FROM FEEDSTOCKS CONTAINING TOXIC ALGAE” and filed on Oct. 10, 2014, which claims the benefit of U.S. Provisional Application Ser. No. 61/889,349 filed on Oct. 10, 2013. Each of the aforementioned references is hereby wholly incorporated by reference in their entirety.

### BACKGROUND

Cyanobacteria are a diverse group of aquatic microorganisms that play significant roles in aquatic ecology. In most aquatic food webs, cyanobacteria are among the primary producers that support the higher trophic levels in the system. In natural aquatic systems such as oceans, lakes and ponds, certain conditions can lead to a rapid increase in the growth of cyanobacteria and eventually a thick overgrowth referred to as a “bloom” can form. Some cyanobacteria found in blooms may produce toxins, and when toxic cyanobacteria are a component of the bloom, the bloom is referred to as a harmful algal bloom (HAB).

Besides toxins, an HAB can have multiple “harmful” features. The toxic cyanobacteria, or indeed any overgrowth of algae, can shade the water, preventing benthic photosynthetic organisms from getting the sunlight they need for growth and survival. Due to the sheer volume of the cyanobacterial biomass, the system can become depleted of oxygen, because oxygen is used by the heterotrophic bacteria that degrade the cyanobacteria as they die. This rapid oxygen consumption can lead to the death of oxygen-dependent plants and animals in the system. “Fish kills” are a common consequence of HABs, due to oxygen depletion that leads to the suffocation of the fish, for example. For these reasons and others, HABs are not viewed as a useful resource, but rather as an ecological threat that needs to be prevented and/or eradicated.

However, an HAB may contain beneficial or potentially beneficial metabolites yet to be discovered, including metabolites that may have application in personal care products. Further, the volume of cyanobacteria that can be accessed in an HAB may be well beyond the volume of cyanobacteria that can be obtained through culturing in laboratories or constructed growth systems. HABs therefore present an opportunity for obtaining large quantities of cyanobacteria for useful purposes, without having to deliberately culture the cyanobacteria.

While these benefits or potential benefits have been recognized by the inventors, the nature of HABs presents many challenges for use in personal care applications. One challenge has already been mentioned above: certain cyanobacteria in an HAB, termed herein as “toxic cyanobacteria,” produce toxins.

Certain toxins would have to be removed, or reduced to a non-harmful concentration in any preparation that relates to personal care products. If the preparation is to be used in skin care products, for example, it must be free of dermatotoxins, or the preparation must have a concentration of dermatotoxins that is reduced to a safe level for use.

A second challenge is the heterogenous nature of an HAB. Typically in a bioprocess aimed to lead to a commercial product, one seeks to work with a culture of a specific type of organism, or a limited and controlled scope of organisms. An HAB, by contrast, is typically an assemblage of many types of algae, nontoxic cyanobacteria, zooplankton such as Daphnia, and bacteria, in addition to the toxic cyanobacteria. As collected directly from its natural source, the bloom material may further contain non-biological components such as silt and sediment. Heterogenous material is non-ideal for a bioprocess from which preparations for use in personal care products may be derived, because heterogeneity makes it more difficult to obtain preparations with consistent properties that are required for the application.

A third and similar challenge to the one above is that, unlike growth of organisms in a controlled system or a constructed growth system, growth of an HAB is not reproducible, meaning that the biomass is never exactly replicated in the natural system where it is found. This again is a non-ideal scenario for the purpose of a bioprocess leading to preparations for use in personal care products, because it makes it difficult to obtain consistent preparations. Further, reproducibility of the biomass is typically required for methods or processes to be commercially viable.

### OBJECTS OF THE INVENTION

One main object of the subject innovation is to create a preparation that can be used in personal care products, and which is derived from biomass from a naturally-grown HAB containing toxic cyanobacteria. Another object of the subject innovation is to disclose methods for obtaining the preparation and characterizing its suitability for use in personal care products. Aspects of the innovation described herein include components of an integrated system enabling execution of the methods. Another object of the subject innovation is to describe methods for use of the biomass-derived preparation in personal care products.

### BRIEF OVERVIEW OF THE INVENTION

This brief overview is provided to introduce a selection of concepts in a simplified form that are described in the detailed description that follows. As such, this brief overview is not intended to be an extensive overview of the claimed subject matter, to identify key factors or essential features of the claimed subject matter, nor is it intended to be used to limit the scope of the claimed subject matter.

Herein, the term “biomass” refers to all the biological and non-biological components that may be collected from a naturally-occurring bloom. As will be described in detail, it is to be understood and appreciated that the biomass, and therefore the bloom, often contains toxic cyanobacteria.

A “bloom” is defined herein as an assemblage containing most any of, but not limited to, the following: algae, nontoxic cyanobacteria, zooplankton, bacteria, and toxic cyanobacteria. In most scenarios, a bloom is defined as having originated in a natural aquatic system.

Examples of toxic cyanobacteria include, but are not limited to, members of the genera Anabaena, Cylindrospermopsis, Dolichospermum, Lyngbya, Nodularia, Hapalosiphon, Nostoc, Oscillatoria, Planktothrix, Raphidiopsis, Schizothrix, Trichodesmium, and Umezakia.

“The preparation” is defined herein as the product of a series of process steps applied to biomass, and which is intended for use in personal care products.

Described herein are systems, methods and embodiments associated with the use of biomass for generating the preparation. The methods and embodiments often include collecting evidence of the presence of toxic cyanobacteria within biomass. It is not a requirement that the biomass was collected for this specific purpose; it may have been collected for any purpose or application.

Upon identification of biomass containing toxic cyanobacteria, the systems, methods and embodiments described herein include process steps applied to the biomass, and which will ultimately lead to the preparation. Process steps may include, but are not limited to: subjecting the biomass to physical or chemical treatments that will disrupt cell walls of the cyanobacteria, followed by separating the cellular components on the basis of density into high and low-density fractions, separating molecules within the low-density fractions on the basis of size to generate fractions that are classified by the relative sizes of the molecules they contain, separating molecules within the size-fractionated components on the basis of chemistry, and performing a nanoseparation step that relies upon properties of size, chemistry and stability to generate unique fractions. A process step may include subprocesses that are not specified, but which are known to those skilled in the art.

The methods and embodiments described herein may include tests and analyses used to assess the outcomes of process steps. Examples of tests and analyses which may be performed, but which are neither required in all cases nor limiting of the types of tests that can be performed, are: biochemical tests for antioxidants, examination of the liquid by ultraviolet-visible spectroscopy, and tests of the liquid's ability to prevent damage to DNA (deoxyribonucleic acid).

The methods and embodiments described herein may include the addition of chemicals at certain process steps, and/or to the preparation that is to be used in personal care products. Examples of chemicals which may be added, and which are neither required in all cases nor limiting of the types of chemicals that can be added, are: flocuulants to aid in density-based separations, preservatives that prevent microbial growth, antioxidants, solvents, and additional ingredients that have application for personal care products. Further, the chemicals which may be added may be either synthetic or natural in origin.

The process steps to which the biomass has been subjected will ultimately yield the preparation. Methods and embodiments are described for use of the preparation in a skin care product. Skin care products are one type of personal care product to which the preparation may be added, and the scope of use of the preparation is not limited to skin care products.

The following description and drawings set forth certain illustrative aspects and implementations. These are indicative of but a few of the various ways in which one or more aspects may be employed. Other aspects, advantages, or novel features of the disclosure will become apparent from the following detailed description when considered in conjunction with the drawings.

### BRIEF DESCRIPTION OF THE DRAWINGS

The accompanying drawings, which are incorporated in and constitute a part of the specification, illustrate various systems, methods, and other embodiments of the disclosure. Illustrated element boundaries (e.g., boxes, groups of boxes, or other shapes) in the figures represent one example of the boundaries. In some examples one element may be designed as multiple elements or multiple elements may be designed as one element. In some examples, an element shown as an internal component of another element may be implemented as an external component and vice versa.

FIG. 1 illustrates a an example method associated with use of biomass for personal care products.

FIG. 2 illustrates an example method associated with use of biomass for personal care products, and within that method subprocesses for the detection of toxic cyanobacterial.

FIG. 3 illustrates an example method associated with use of biomass as defined herein for personal care products, and within that method specific subprocesses for generating the preparation for use in personal care products.

FIG. 4 illustrates an example system with three primary components that can be integrated in order to carry out the methods and embodiments described.

FIG. 5 illustrates an example Analytical Component of the integrated system that was illustrated in FIG. 4.

FIG. 6 illustrates an example Biochemical Activity Component in accordance with the embodiment illustrated in FIG. 5.

FIG. 7 illustrates an example Human Subjects Component in accordance with the embodiment illustrated in FIG. 5.

FIG. 8 illustrates an example Preparation Production Component in accordance with the embodiments of FIG. 4.

FIG. 9 illustrates an example Fractionation Component in accordance with embodiment of FIG. 8.

### DETAILED DESCRIPTION

Embodiments or examples illustrated in the drawings are disclosed below using specific language. It will nevertheless be understood that the embodiments or examples are not intended to be limiting. Any alterations and modifications in the disclosed embodiments, and any further applications of the principles disclosed in this document are contemplated as would normally occur to one of ordinary skill in the pertinent art. Described herein are examples of methods and embodiments associated with obtaining products from biomass as it has been defined herein.

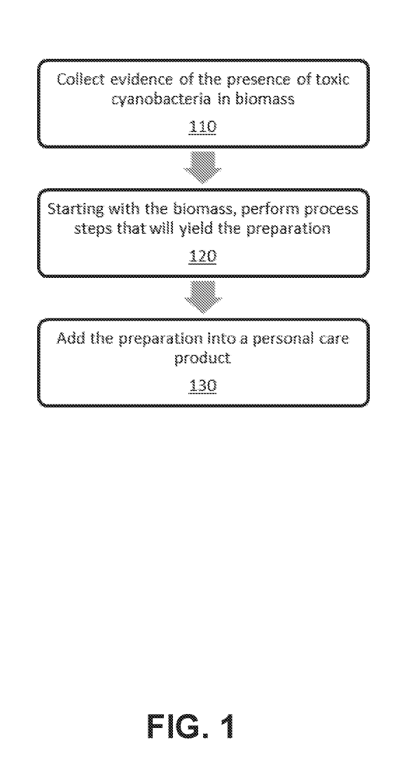

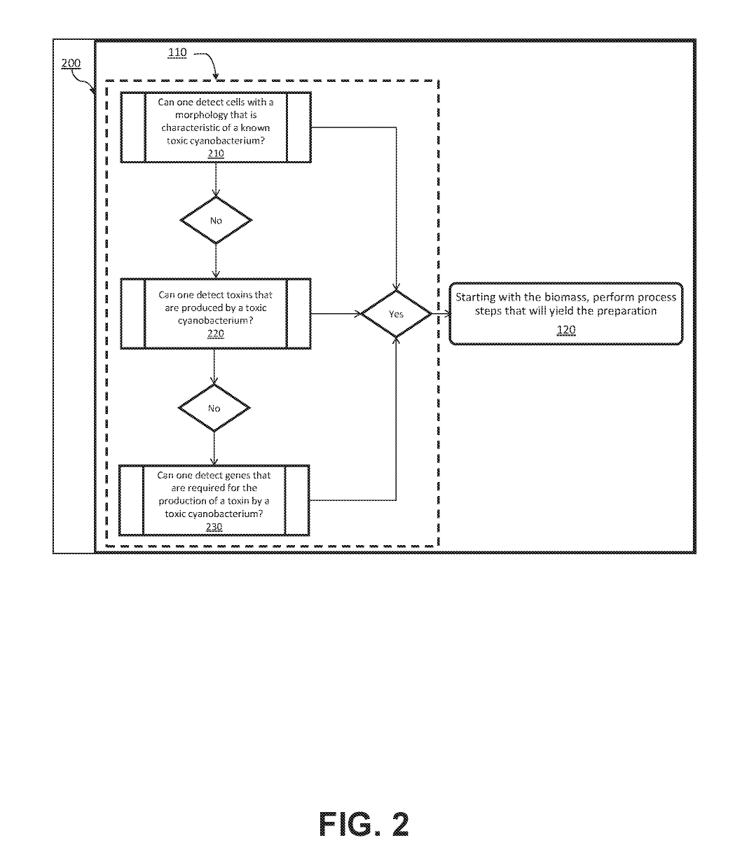

FIG. 1 is an illustration of an example method associated with the use of biomass for personal care products. The biomass may have been collected for this or most any other purpose. At 110, evidence is collected for the presence of toxic cyanobacteria in biomass. If toxic cyanobacteria are not detected, the method does not proceed to 120. Collection of evidence will be described in greater detail with reference to FIG. 2 infra.

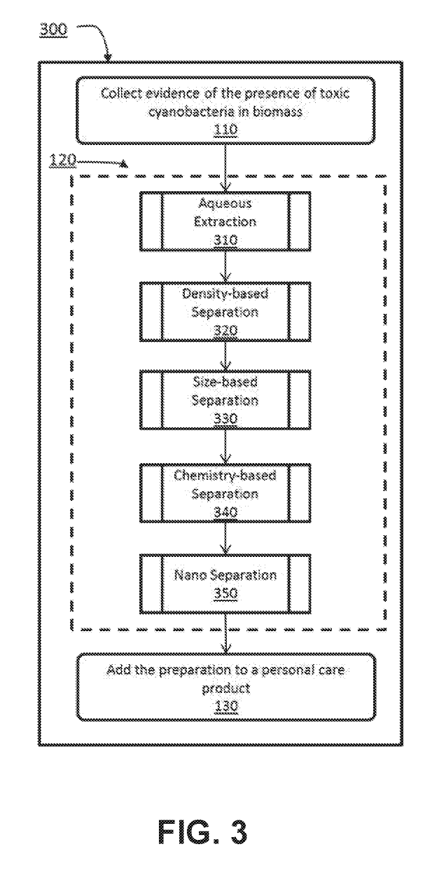

If evidence of the presence of toxic cyanobacteria is collected, the method proceeds to 120. At 120, processes and subprocesses will be performed that will yield the preparation, as detailed in FIG. 3 infra. At 130, the preparation that results from 120 will be used in a personal care product. The personal care product is expected to comprise numerous ingredients in addition to the preparation. While the examples described herein are specifically directed to the use of the preparation in personal care products, it is to be understood and appreciated that the scope of this specification is not intended to be limited to such uses. Accordingly, additional end uses that employ the features, functions and benefits of this innovation are to be included within the scope of this specification and claims appended hereto.

FIG. 2 is an illustration of a method 200 for collecting evidence for the presence of toxic cyanobacteria in biomass, and then proceeding on to process the biomass into the preparation. At 210, one would identify whether cells that are representative of a toxic cyanobacterium are present. In one embodiment, light microscopy is used at 210. In another embodiment, fluorescence microscopy is used at 210. It is to be understood and appreciated that, in addition to those specifically described, most any techniques known, or that become known, in the art can be employed to detect toxic cyanobacteria without departing from the spirit and scope of the innovation.

If cells of any toxic cyanobacterium are found to be present, or if cells appear to be those of a toxic cyanobacterium but may not be confirmed due to poor resolution or a similar limitation are found to be present, the method continues to 120. If no cells of a toxic cyanobacterium appear to be present, the method proceeds to 220. At 220, common methods used for detection of toxins produced by toxic cyanobacteria may be employed. In one embodiment, an enzyme-linked immunosorbent assay may be used to identify a toxin. In another embodiment, a mouse bioassay may be used to identify a toxin. In another embodiment, high-pressure liquid chromatography (HPLC) combined with mass spectroscopy (MS) may be used to identify a toxin. If any toxin is detected, the method continues to 120. If no toxins are detected, the method continues to 230.

At 230, molecular biological tools may be employed to detect the presence of genes or gene products involved in the production of toxins that are associated with toxic cyanobacteria. In one embodiment, quantitative polymerase chain reaction may be used at 230 to detect genes. In another embodiment, sandwich hybridization may be used at 230 to detect genes. In yet other embodiments, reverse-transcriptase polymerase chain reaction may be used to detect ribonucleic acid gene products. If molecular biological tools detect the presence of any genes or gene products involved in the production of toxins that are associated with toxic cyanobacteria, the method continues on to 120. If no genes or gene products are detected, the method is presumed to terminate.

At 120, the biomass undergoes process steps that will yield the preparation. FIG. 3 is an illustration of one possible set of process steps that could yield the preparation. With the biomass from 110, an aqueous extraction can be performed at 310. In one embodiment, an aqueous extraction involves freezing and then thawing the biomass at least one time. In another embodiment, ultrasonication of the biomass in water may be employed for aqueous extraction. These are provided as examples; different techniques may be employed which are known to lyse toxic cyanobacteria so that they will release their cellular contents into the aqueous phase. In the embodiment described here, an aqueous extract is generated as an output, and at 320 the aqueous extract generated at 310 will undergo separation into fractions on the basis of density.

At 320 numerous processes and subprocesses are options for density-based separations. In one embodiment centrifugation may be used. In another embodiment, settling out of denser components may be used. In order to alter the densities of the constituent components of the aqueous extract, flocculants or chemical additives may be added so that separation via settling occurs in a way that leaves fewer microsolids in the liquid supernatant that will be collected. In another embodiment, both flocculation and centrifugation may be used. For example, flocculation can yield a liquid from which larger, denser solids have been removed but which still contains microsolids. Through centrifugation, such microsolids may be further removed so that the output of this process step is a well-clarified liquid. Of relevance for 320 is that a solid or multiple solid fractions will be generated which may be used for purposes beyond the scope of this method or discarded, while a liquid fraction will be generated as the output for further processing within the scope of this method.

At 330 the liquid output of 320 will undergo a size-based separation that will separate retained microsolids and large molecules from small molecules that are the targets for inclusion in the preparation. In most embodiments of 330, membranes will be used which have a molecular weight cutoff that will lead to the retention of suspended microsolids and large molecules (the “large” class), and the flowthrough of small molecules (the “small” class). In one embodiment, basic filtration using cellulose-based membranes may be employed to separate the large class from the small class. In another embodiment, a polyethersulphone tubular membrane may be used. In another embodiment a polysulphone membrane is used. In another embodiment, a polyvinylidene fluoride membrane is used. In yet another embodiment, a tangential flow filtration membrane is used.

As an example of an embodiment where a tangential flow filtration membrane is used, the molecular weight cutoff associated with the membrane is 10 kDa. In this example, the large class comprises microsolids and molecules greater than 10 kDa in size. The tangential flow filtration retentate containing the large class will be collected and preserved for future processes that are beyond the scope of this method. A liquid fraction containing the small class, comprised of molecules less than 10 kDa in size, will be generated as the output for further processing within the scope of this method.

At 340 the liquid containing the small class will be subjected to a separation based upon the chemistry of the molecules that are desired to be retained for the preparation. In one embodiment, a liquid-liquid extraction utilizing a nonpolar solvent such as phenol is used. The hydrophobic molecules retained in the nonpolar solvent will be preserved for future processes that are beyond the scope of this method. The hydrophilic molecules retained in the aqueous phase would be collected as the output of 340 and carried on to 350. In another embodiment, a silica-based resin modified with C-18 residues is used, wherein hydrophobic molecules will be retained by the resin, and hydrophilic molecules will pass through the resin, under specific conditions related to the liquid milieu in which the molecules are contained. The molecules retained by the resin can be eluted with a nonpolar solvent, and will be preserved for future processes that are beyond the scope of this method. The molecules that passed through the column will have been collected as the output of 340. In one example of this embodiment, reverse-phase column chromatography is used. Reverse-phase column chromatography is well known to those skilled in the art, and what is unique about its use here is that it serves as a “scrubber” to remove molecules that are not desired or not necessary for the preparation. Such molecules could include the toxins produced by toxic cyanobacteria that were present in the original biomass, and as identified at 110 of FIGS. 1 and 2 infra. However, removal of toxins is only one possible benefit of this step. Process step 340 enables the preparation that will be used at 130 to be more refined, safer, and more consistent than any other preparation intended for use in personal care products and which is derived from naturally-occurring bloom material that may contain toxic cyanobacteria. This step in part addresses the challenge presented by inconsistency in the biomasses that may be used, as mentioned in the Background section.

At 350, the hydrophilic molecules that were collected at 340 will undergo Nano Separation both to further refine and to concentrate the desired molecules into the preparation. In one embodiment, this is accomplished using a polyamide film tubular nanofiltration membrane to separate the hydrophilic molecules into a retentate and a flowthrough. The retentate will undergo subprocessing, such as the addition of preservatives or stabilizers, to yield the preparation that will be used at 130. As was the case with the reverse-phase chromatography example described for process step 340, nanofiltration is well-known to those skilled in the art. However, it has never been applied to the output of process steps such as those described in 340, in a manner that yields the preparation intended for use in personal care products and which is derived from naturally-occurring bloom material that may contain toxic cyanobacteria. This is highly significant because the use of biomass from naturally-occurring blooms for personal care products has been limited by issues related to refinement, safety and consistency. Through the method and embodiments described above, the preparation overcomes these limitations in an economically-viable fashion, and has the added benefits of being enriched for molecules that are highly desirable in personal care products.

At 130, the preparation will be added to a formulation intended for use as a personal care product, or other product as on desired. Examples of personal care products include but are not limited to cleansers, toners, lotions, creams, sera, and liquid foundations. For example, in one embodiment, the personal care product is an anti-aging serum to which the preparation is added at a concentration of 2% (w/w). In another embodiment, the personal care product is a hair conditioner to which the preparation is added at a concentration of 4% (w/w).

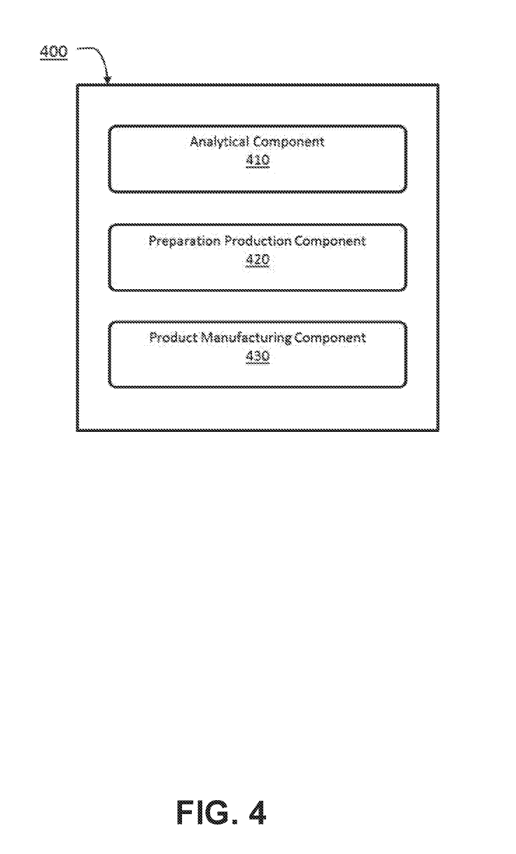

All of the embodiments described above for FIGS. 1, 2 and 3 are listed as example techniques; different techniques may be used to separate the constituents of the biomass and to use the preparation. Further, the overall method is not restricted to any specific volumes or scale of operation, and in fact recognizes that at different scales minor modifications to the basic process steps may be required. It is to be understood that any process step or act may require specific conditions, such as temperature, pressure, and pH, to yield desired outputs. Modification of such conditions may in fact enhance the recovery of some outputs relative to others, and such enhancements are monitored via the Analytical Component shown at 410 in FIG. 4.

FIG. 4 illustrates one embodiment of a system associated with making personal care products that incorporate the preparation. At 410 is shown the Analytical Component. This Component is used in conjunction with both the Preparation Production Component, 420, and the Product Manufacturing Component, 430. As will be described below, 420 and 430 encompass the methods, embodiments, and examples which have been described previously in this document, as well as additional methods, embodiments, and examples. Within most of those methods, embodiments and example analyses are performed to monitor inputs and outputs, and the fate of molecules of interest for the preparation. Likewise, the preparation may undergo analyses to monitor its safety and consistency.

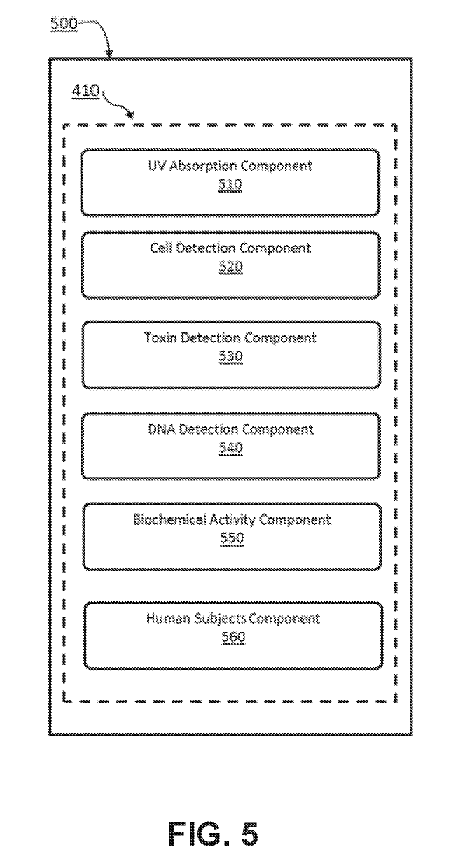

FIG. 5 illustrates examples of components that may be part of the Analytical Component shown at 410. At 510 is found a component for measuring ultraviolet (UV) absorption. This component may be used, for example, at 310 through 350 shown in FIG. 3, so that molecules that absorb wavelengths of light in the ultraviolet range may be selectively carried on to subsequent process steps. This component may also be used, for example, to measure concentrations of DNA, ribonucleic acid (RNA), amino acids, proteins, and vitamins.

In one embodiment, the UV Absorption Component at 510 comprises a spectrophotometer. In another embodiment, 510 comprises HPLC with an in-line photodiode array. In accordance with this embodiment, the output with the highest absorbance at 333 nm is deemed the most suitable for use in each subsequent process, and ultimately for the preparation to be added to personal care products at 130. Alternatively, the techniques may be used in conjunction with one another.

Within FIG. 5, the Cell Detection Component at 520 may be configured to include light microscopy, fluorescence microscopy, fluorometers, or similar tools that can be used to assess for the presence or absence of cells of toxic cyanobacteria. Note that a single or multiple techniques may comprise 520 so that one may draw a reasonable conclusion enabling the progression from 210 to 120, as shown in FIG. 2. The Toxin Detection Component at 530 may be configured to include enzyme-linked immunosorbent assays with a microtiter plate reader, HPLC with an in-line photodiode array, MS, or UV spectroscopy. A single or multiple techniques may comprise 530 so that one may draw a reasonable conclusion enabling the progression from 220 to 120, as shown in FIG. 2. The DNA Detection Component at 540 may be configured to include Southern blots, Northern blots, polymerase chain reaction, or quantitative polymerase chain reaction to collect evidence for the presence of DNA associated with the production of toxins by toxic cyanobacteria. A single or multiple techniques may comprise 530 so that one may draw a reasonable conclusion enabling the progression from 230 to 120, as shown in FIG. 2.

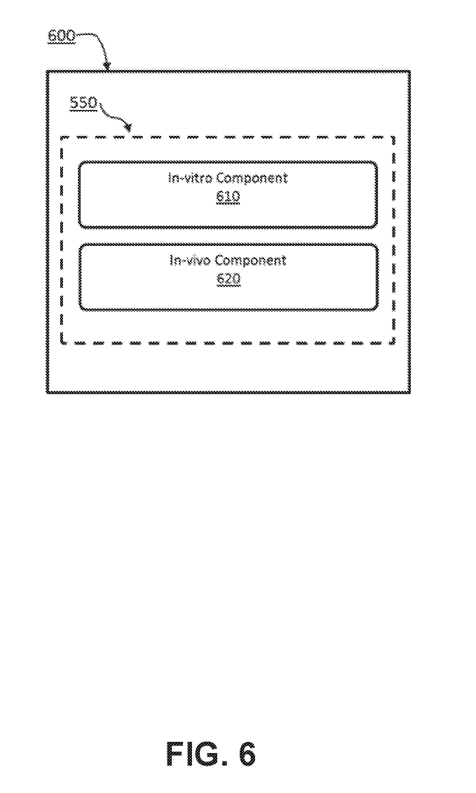

At 550 the Biochemical Activity Component may be configured to include any techniques or technologies that not only seek to confirm the presence or absence of certain molecules or sets of molecules, but rather the actual biological activity of such molecules or sets of molecules that ultimately are to be included or excluded from the preparation. FIG. 6 illustrates that the Biochemical Activity Component at 550 may be configured to include both or either an In-vitro Component, 610, and an In-vivo Component, 620. Examples of techniques at 610 are assays for the prevention of oxidation of indicator molecules (antioxidant activity), and assays for the prevention of either chemical or UV-induced damage to DNA. Such assays may be used to assess properties of the preparation. Examples of techniques at 620 are cell-based assays for protection against phototoxicity or inflammation, or the use of live animals to assess for the presence of certain toxins, especially those which may originate from toxic cyanobacteria.

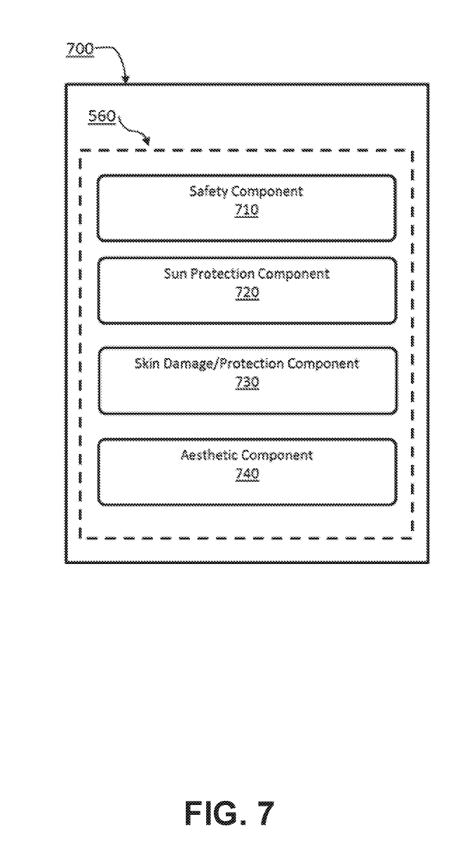

Returning to FIG. 5, Analytical Component 410 may be configured to include a Human Subjects Component, 560. FIG. 7 illustrates that the Human Subjects Component at 560 may be configured to include Safety (710), Sun Protection (720), Skin Damage/Protection (730), and Aesthetic (740) Components. Each of Components 710 through 740 may comprise the use of human subjects and methods widely known to those skilled in the art.

At present, Analytical Component 410 is entirely comprised of methods, techniques and technologies known to those skilled in the art, though it is not limited to presently known methods, techniques and technologies and in the future may be inclusive of better methods, techniques and technologies. The methods, techniques, and technologies that are described herein are of importance because it is the use of such methods, techniques, and technologies for the baseline method of FIG. 1 that enables one to arrive at the preparation. The preparation originates from biomass from naturally-occurring blooms and which contains toxic cyanobacteria. Without the application of such methods, techniques, and technologies to assess progression through the baseline method of FIG. 1, the use of such biomass to generate the preparation specific to this invention would not occur.

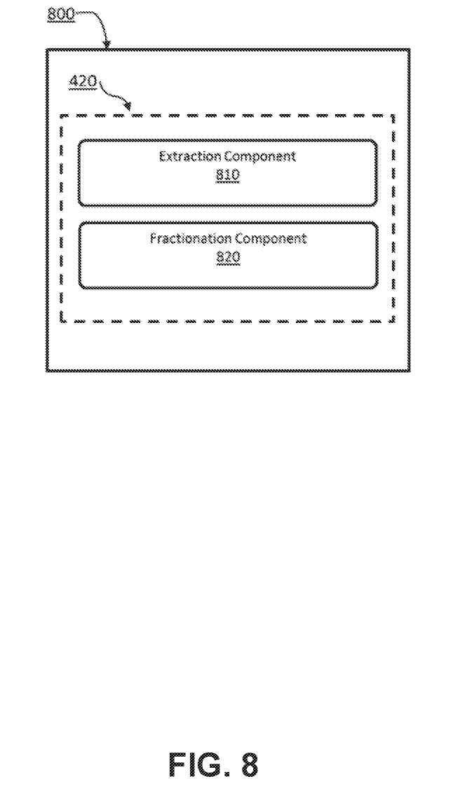

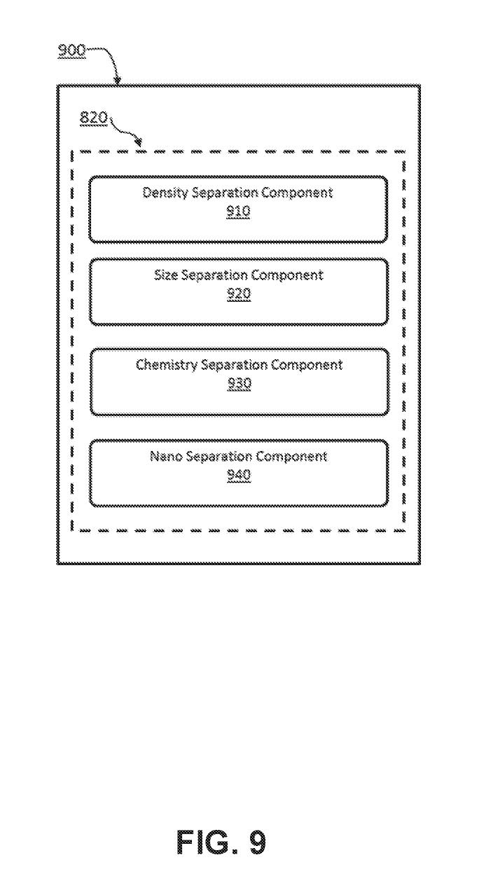

Similarly, the use of such biomass to generate the preparation would not occur without Production Component 420 illustrated in FIG. 4, and further illustrated in FIG. 8. At 420, Extraction Component 810 and Fractionation Component 820 may be configured. At 810 one may find the techniques and technologies associated with Aqueous Extraction that was part of the method, and shown at 310 of FIG. 3. At 820 one may find the techniques and technologies associated with process steps 320 through 350 of FIG. 3. The Fractionation Component, 820, is further detailed in FIG. 9, which illustrates a configuration that includes Components required to execute process steps 320 through 350 of FIG. 3. Note that these Components may be applied for additional process steps or subprocess steps that have not been described, and that the process steps described herein are not intended to limit the method to those process steps only. Similarly, the Components described herein are not intended to limit the System (FIG. 4) to these Components only.

Returning to FIG. 4, the Product Manufacturing Component, 430, may be configured so that the preparation will eventually be incorporated into a finished personal care product that may include not only the preparation but other ingredients that enhance the quality and performance of the product. Use of the preparation within Component 430 would not be possible without Components 410 and 420 and the unique method described herein.

While for purposes of simplicity of explanation, illustrated methodologies are shown and described as a series of blocks. The methodologies are not limited by the order of the blocks as some blocks can occur in different orders and/or concurrently with other blocks from that shown and described. Moreover, less than all the illustrated blocks may be used to implement an example methodology. Blocks may be combined or separated into multiple components. Furthermore, additional and/or alternative methodologies can employ additional, not illustrated blocks. Additionally, it is to be understood that a “component” can refer to hardware, software or a combination of hardware and software as appropriate to carry out the process and methods described herein.

To the extent that the term “includes” or “including” is employed in the detailed description or the claims, it is intended to be inclusive in a manner similar to the term “comprising” as that term is interpreted when employed as a transitional word in a claim.

While example methods, embodiments and so on have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the disclosure is not limited to the specific details, the representative apparatus, and illustrative examples shown and described. Thus, this application is intended to embrace alterations, modifications, and variations that fall within the scope of the appended claims, which satisfy the statutory subject matter requirements of 35 U.S.C. §101.

What is claimed is:

1. A method of manufacturing a personal care product, comprising:
collecting evidence of the presence of toxic cyanobacteria in a biomass;
processing the biomass to yield a preparation; and
manufacturing a personal care product that includes the preparation.

2. The method of claim 1, further comprising:
collecting the biomass, wherein the biomass comprises biological and non-biological components related to a naturally-occurring bloom and wherein the bloom includes toxins;
subjecting the biomass to physical or chemical treatments that disrupt cell walls of the cyanobacteria;
separating the cellular components of the biomass on a basis of density into a high or low density fraction;
separating molecules within the low density fraction based at least upon size, wherein the separation of the molecules generates size-fractioned molecules classified by the relative sizes of the molecules contained therein;
separating a subset of the molecules within the size-fractioned molecules based upon chemistry; or
performing a nanoseparation based upon properties of size, chemistry and stability to generate unique fractions, wherein the unique fractions yield the preparation.

3. The method of claim 1, wherein the collection of the evidence of the presence of toxic cyanobacteria in the biomass comprises one of detecting, via light or fluorescence microscopy, a subset of cells with a morphology that is characteristic of a known toxic cyanobacterium, detecting, via enzyme-linked immunosorbent assay, mouse bioassay or high-pressure liquid chromatography (HPLC), a toxin that is produced by a toxic cyanobacterium or detecting, via quantitative polymerase chain reaction, sandwich hybridization or reverse-transcriptase polymerase chain reaction, genes that are used in production of a toxin by a toxic cyanobacterium.

4. The method of claim 1, wherein processing the biomass to yield the preparation includes at least one of aqueous extraction, density-based separation, size-based separation, chemistry-based separation or nano-separation to yield the preparation.

5. The method of claim 1, further comprising, manufacturing the personal care product by adding at least one of flocuulants that aid in density-based separation, preservatives that prevent microbial growth, antioxidants, or solvents.

6. The method of claim 1, wherein the personal care product is a skin care product.

7. The method of claim 1, wherein the bloom is one that is confirmed to contain toxic cyanobacteria.

8. The method of claim 1, wherein the bloom originates in natural aquatic system.

9. The method of claim 1, wherein the preparation is added to the personal care product at a concentration of about 2% (w/w).

10. A system for manufacturing a personal care product, comprising:
an analytical component that collects evidence of the presence of toxic cyanobacteria in a biomass;
a preparation production component that processes the biomass to yield a preparation; and
a product manufacturing component that facilitates manufacture of a personal care product that includes the preparation.

11. The system of claim 10, wherein the analytical component includes a UV absorption component.

12. The system of claim 11, wherein the analytical component measures concentrations of DNA, ribonucleic acid (RNA), amino acids, proteins and vitamins.

13. The system of claim 11, wherein the UV absorption component comprises a spectrophotometer.

14. The system of claim 10, further comprising a cell detection component that assesses one of presence or absence of cells of toxic cyanobacteria.

15. The system of claim 10, further comprising one of a toxin detection component configured to include enzyme-linked immunosorbent assays with a microtiter plate reader, HPLC with an in-line photodiode array, MS or UV spectroscopy, a DNA detection component configured to include Southern blots, Northern blots, polymerase chain reaction or quantitative polymerase chain reaction that collect evidence of presence of DNA associated with production of toxins via toxic cyanobacteria, or a biochemical activity component that confirms presence or absence of certain molecules related to the preparation.
